(19) United States
(12) Patent Application Publication
(10) Pub. No.: US 2023/0099710 A1
(43) Pub. Date: Mar. 30, 2023

(54) VASCULAR ACCESS REVERSING DEVICE AND METHODS OF USE THEREOF

(71) Applicant: Joseph A. Ramirez-Cárdenas, Yuma, AZ (US)

(72) Inventors: Joseph A. RAMÍREZ-CÁRDENAS, Yuma, AZ (US); James P. HOPE, Nashville, TN (US); Kimberly A. LEON, Old Hickory, TN (US); Michael A. KAMM, Thompsons Station, TN (US); Jordan R. MYKLEBY, Nashville, TN (US); Michael AUGUSTINE, Nashville, TN (US)

(21) Appl. No.: 17/936,243

(22) Filed: Sep. 28, 2022

Related U.S. Application Data
(60) Provisional application No. 63/261,768, filed on Sep. 28, 2021.

(51) Int. Cl. A61M 25/06; A61M 25/00

(52) U.S. Cl. CPC A61M 25/0668; A61M 25/0068

(57) ABSTRACT

A method, system and apparatus for performing an interventional procedure, the method includes retrograde advancement of a first guidewire into the femoral artery and advancing a sheath system over the guidewire and inserting the distal end of a sheath system through an incision and into the femoral artery of the patient. The sheath system comprises a retrograde sheath having an antegrade sheath removably connected thereto, wherein the retrograde sheath comprises a lumen extending between a retrograde sheath distal end and a retrograde sheath proximal end and wherein the antegrade sheath comprises a lumen extending between an antegrade sheath distal end and an antegrade sheath proximal end, the access sheath system further comprising separable section connecting the retrograde sheath to the antegrade sheath. The method further includes separating the retrograde sheath from the antegrade sheath via the tear away section, and performing an antegrade interventional procedure.

### CROSS-REFERENCE TO RELATED APPLICATION

This application claims priority under 35 U.S.C. § 120 of U.S. Provisional Application No. 63/261,768, entitled VASCULAR ACCESS REVERSING DEVICE AND METHODS OF USE THEREOF, filed on Sep. 28, 2021, the disclosure of which is incorporated by reference herein.

### BACKGROUND

### I. Field

This disclosure relates generally to a vascular access reversing device, and more particularly to an apparatus and a method for providing access in an antegrade direction in a vascular environment.

### II. Description of Related Art

The treatment of vascular diseases has grown significantly in terms of sophistication and diversity. Procedures involving items such as stents and balloons are virtually routine in many health-care practices. One issue associated with any vascular procedure relates to how to provide for optimal access to anatomical sites.

Vasculature procedures generally require accessing vasculature via an access site and routing subsequent medical devices through the access site into the patients vasculature via a vein or artery. In more detail, many procedures begin with retrograde access of the superficial femoral artery (“SFA”) through an access site at the common femoral artery (“CFA”), and then passing subsequent medical devices through the SFA from the CFA. In one such method for introducing a catheter into the SFA, a vascular access needle is inserted in a retrograde (i.e., against the flow of blood) into the CFA, and a guidewire is inserted through the vascular access needle and manipulated to the SFA. The vascular access needle is then removed, and an introducer is routed about the guidewire into the CFA and SFA. Finally, the guidewire is removed and a catheter or other medical device is routed through the introducer to perform one or more portions of the medical procedure. However, the Inventor has recognized that the ability to transition between retrograde and antegrade delivery modes, provides several advantages, examples of which are described in further detail below.

### SUMMARY

The following aspects and aspects thereof are described and illustrated in conjunction with systems, tools and methods which are meant to be exemplary and illustrative, not limiting in scope.

In one aspect, a method for performing an interventional procedure is disclosed. The method includes retrograde placement of a needle into the femoral artery of a patient to create an incision, retrograde advancement of a first guidewire in to the femoral artery, and advancing a sheath system over the guidewire and inserting the distal end of a sheath system through the incision and into the femoral artery of the patient. The sheath system comprises a retrograde sheath having an antegrade sheath removably connected thereto, wherein the retrograde sheath comprises a lumen extending between a retrograde sheath distal end and a retrograde sheath proximal end and wherein the antegrade sheath comprises a lumen extending between an antegrade sheath distal end and an antegrade sheath proximal end, the access sheath system further comprising a connection section connecting the retrograde sheath to the antegrade sheath. The method further includes antegrade advancement of a second guidewire into the femoral artery via the lumen of the antegrade sheath, separating the retrograde sheath from the antegrade sheath via the connection section, and performing an antegrade interventional procedure.

In another aspect, a system for performing an interventional procedure is disclosed, the system may include a retrograde sheath having an antegrade sheath removably connected thereto, wherein the retrograde sheath comprises a lumen extending between a retrograde sheath distal end and a retrograde sheath proximal end and wherein the antegrade sheath comprises a lumen extending between an antegrade sheath distal end and an antegrade sheath proximal end. The system may further include a connection section connecting the retrograde sheath to the antegrade sheath, wherein the connection section is configured to allow for separation of the antegrade sheath from the retrograde sheath.

In addition to the example aspects and aspects described above, further aspects and aspects will become apparent by reference to the drawings and by study of the following detailed description.

### BRIEF DESCRIPTION OF THE DRAWINGS

Example aspects are illustrated in the drawings. It is intended that the aspects and figures disclosed herein are to be considered illustrative rather than restrictive.

FIG. 1 is a side schematic view of a system according to one aspect of the disclosure;

FIG. 2 is a side schematic view of the system shown in FIG. 1 in a second state of use according to an aspect of the disclosure;

FIG. 3 is a side schematic view of the system shown in FIGS. 1 and 2 in a third state of use according to an aspect of the disclosure;

FIG. 4A is a side schematic view of the system shown in FIGS. 1-3 in a fourth state of use according to an aspect of the disclosure;

FIG. 4B is a schematic cross-section view taken along lines FF in FIG. 4A;

FIG. 5 is a side schematic view of the system shown in FIGS. 1-4 in a fifth state of use according to an aspect of the disclosure;

FIG. 6 is a side schematic view of the system shown in FIGS. 1-5 in a sixth state of use according to an aspect of the disclosure;

FIG. 7 is a side schematic view of a system according to one aspect of the disclosure;

FIG. 8 is a side schematic view of the system shown in FIG. 7 in a second state of use according to an aspect of the disclosure;

FIG. 9 is a side schematic view of the system shown in FIGS. 7 and 8 in a third state of use according to an aspect of the disclosure;

FIG. 10A is a side schematic view of the system shown in FIGS. 7-9 in a fourth state of use according to an aspect of the disclosure;

FIG. 10B is a schematic cross-section view taken along lines RR in FIG. 10B; and

FIG. 11 is a side schematic view of the system shown in FIGS. 7-10 in a fifth state of use according to an aspect of the disclosure;

FIG. 12 is a side schematic view of a system according to one aspect of the disclosure;

FIG. 13 is a side schematic view of the system shown in FIG. 12 in a second state of use according to an aspect of the disclosure;

FIG. 14 is a side schematic view of the system shown in FIGS. 12 and 13 in a third state of use according to an aspect of the disclosure;

FIG. 15A is a side schematic view of the system shown in FIGS. 12-14 in a fourth state of use according to an aspect of the disclosure;

FIG. 15B is a schematic cross-section view taken along lines RR in FIG. 15A;

FIG. 16 is a side schematic view of the system shown in FIGS. 12-16B in a fifth state of use according to an aspect of the disclosure;

FIG. 17A shows one example of a sheath system according to aspects of the disclosure;

FIG. 17B shows one example of a retrograde sheath according to aspects of the disclosure;

FIG. 17C shows one example of an antegrade sheath according to aspects of the disclosure;

FIGS. 18A-18F show various views of one example of a retrograde sheath and details thereof according to aspects of the disclosure;

FIGS. 19A-19E show various views of one example of the sheath system and details thereof according to aspects of the disclosure; and

FIGS. 20A-20B show various views of one example of the sheath system and details thereof according to aspects of the disclosure.

### DETAILED DESCRIPTION

In the following detailed description, reference is made to the accompanying figures, which form a part thereof. In the figures, similar symbols typically identify similar components, unless context dictates otherwise. The illustrative aspects described in the detailed description, figures, and claims are not meant to be limiting. Other aspects may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

The following includes definitions of selected terms employed herein. The definitions include various examples and/or forms of components that fall within the scope of a term and that may be used for implementation. The examples are not intended to be limiting. Further, it will be obvious to one skilled in the art that the present disclosure may be practiced without these specific details. In other instances, well-known methods, procedures, and components have not been described in detail so as to not unnecessarily obscure aspects of the present disclosure.

Throughout the disclosure, the term substantially or approximately may be used as a modifier for a geometric relationship between elements or for the shape of an element or component. While the terms substantially or approximately are not limited to a specific variation and may cover any variation that is understood by one of ordinary skill in the art to be an acceptable variation, some examples are provided as follows. In one example, the terms substantially or approximately may include a variation of less than 10% of the dimension of the object or component. In another example, the terms substantially or approximately may include a variation of less than 5% of the object or component. If the terms substantially or approximately are used to define the angular relationship of one element to another element, one non-limiting example of the terms may include a variation of 5 degrees or less. These examples are not intended to be limiting and may be increased or decreased based on the understanding of acceptable limits to one of ordinary skill in the art.

Throughout the disclosure, when reference is made to the terms proximal or proximally it is intended to mean a portion or component of the system that is oriented away from the body into which the system is or is intended to be placed. Conversely, when reference is made to the terms distal or distally it is intended to mean a portion or component of system that is oriented toward the body into which the system is or is intended to be placed. Thus, for example, the guiding atraumatic tip described hereinafter is located at a distal end of the sheath system, while the proximal hub is located at a proximal end of the sheath system.

Throughout the disclosure, the system, apparatus, and method in accordance with the disclosure is described in the context of access to the common femoral artery (“CFA”) and superficial femoral artery (“SFA”), it will be understood that the sheath or catheter system may be used clinically for a variety of different therapeutic or diagnostic indications involving vascular interventions, including, for example and without limitation, vascular occlusion, angioplasty, stent delivery, artherectomy, drug delivery, imaging or the like. For example, applications of the sheath or catheter system may involve access at the brachial artery, the subclavian artery, or any other blood vessel suitable for use as an access site for catheterization, including venous vessels.

For purposes of the disclosure, directional terms are expressed generally with relation to a standard frame of reference when the described device is in an in-use orientation.

Example aspects of the disclosure offering one or more of the advantages described below include a system, vascular access device, and method of use thereof. The disclosed aspects facilitate performance of peripheral vascular intervention procedures, and allows for transitions between retrograde and antegrade access without generation of multiple access sites and without unnecessary or excessive vascular damage.

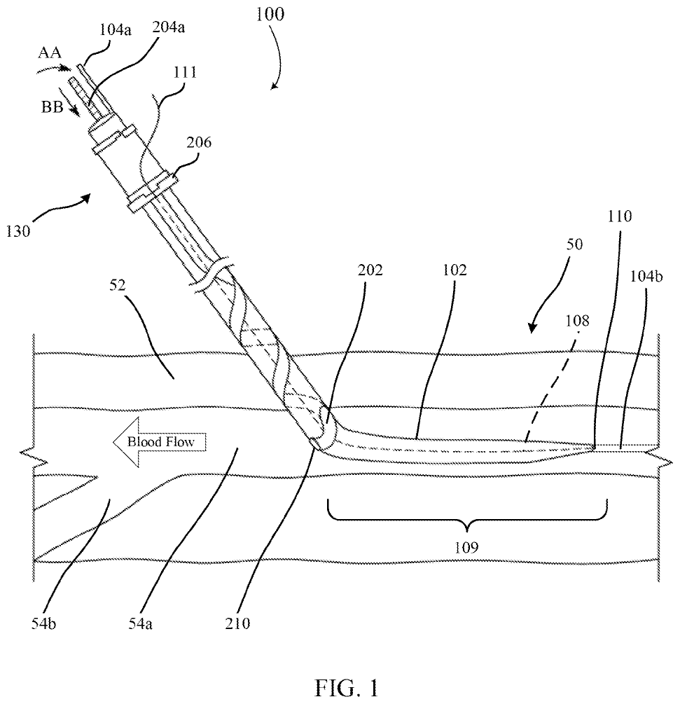

FIG. 1 shows one example of a catheter or sheath in accordance with one aspect of the disclosure. It is noted that throughout the disclosure, the terms sheath and catheter may be used interchangeably. In the Example shown in FIG. 1, a sheath system 100 may be inserted in the common femoral artery (“CFA”) 54a through the tissue 52 of a patient 50. The sheath system 100 may be placed retrograde going toward the heart (i.e. against the natural direction of the bloodstream). The sheath system 100 may for example include a retrograde sheath 102 having an interior lumen 110 and an antegrade sheath 202 having an interior lumen 210. As shown in FIG. 1, the antegrade sheath 202 may be braided and/or helically disposed around the lumen of the retrograde sheath 102. As described in further detail below, the antegrade sheath 202 may be removably connected to or otherwise separable from the retrograde sheath 102. In one example aspect, the retrograde sheath 102 may for example have connection section 108 connected to a tear away activator 111. The connection section 108 may be a perforable or tear away section that is capable of separating or otherwise causing a separation or break between the inner lumen and the outer wall of the retrograde sheath 102 in response to an activation (e.g., a user pulling) the activator 111 and/or in response to a user retracting the retrograde sheath 102 with respect to the antegrade sheath 202. Further description of the connection section 108 and activator 111 are described in further detail below.

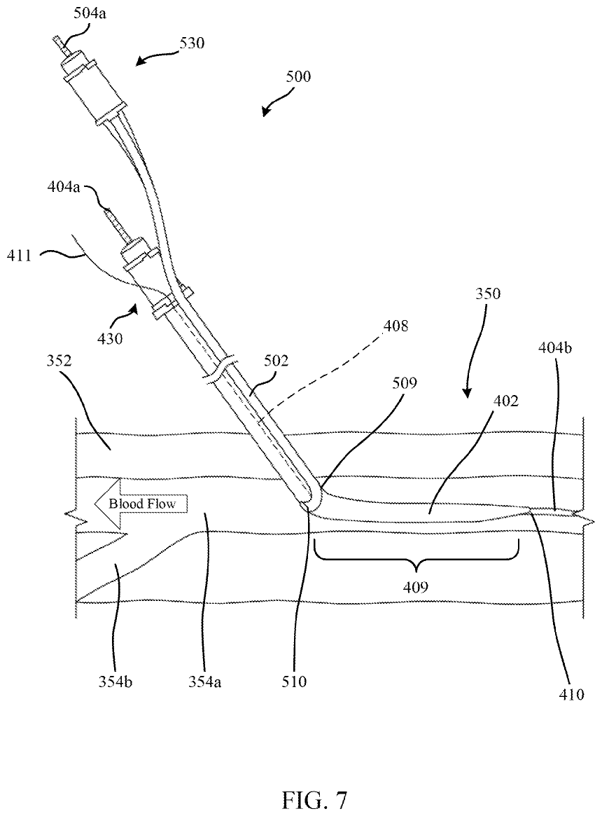

The sheath system may include a hub 130 with one or more hemostasis valve(s) as a first hemostasis valve portion and a second hemostasis valve portion. As an alternative, and as described in further detail below respect to FIGS. 7-11, as an alternative to a single hub 130 with multiple hemostasis valve portions, each sheath of the sheath system may have a respective hub with that comprises a hemostasis valve. As shown in FIG. 1, the hemostasis valve 130 may for example comprise a separate hemostasis valve portion for each of the retrograde sheath 102 and the antegrade sheath 202. In an alternative aspect, the hemostasis valve 130 may instead be an interface, such as a threaded interface that is configured to engage with (e.g., thread onto) or otherwise engage with a separate hemostasis valve (which may for example be an off-the-shelf or otherwise known hemostasis valve(s)). In one example, each of the two hemostasis valve portions of the hemostasis valve 130 may be configured to receive a respective one of a retrograde guidewire 104a and/or an antegrade guidewire 204a. While not shown in the figures, each one or both of the hemostasis valve portions of the hemostasis valve 130 may for example include a sheath flush line with one or more valves and flush fitting(s) at ends thereof.

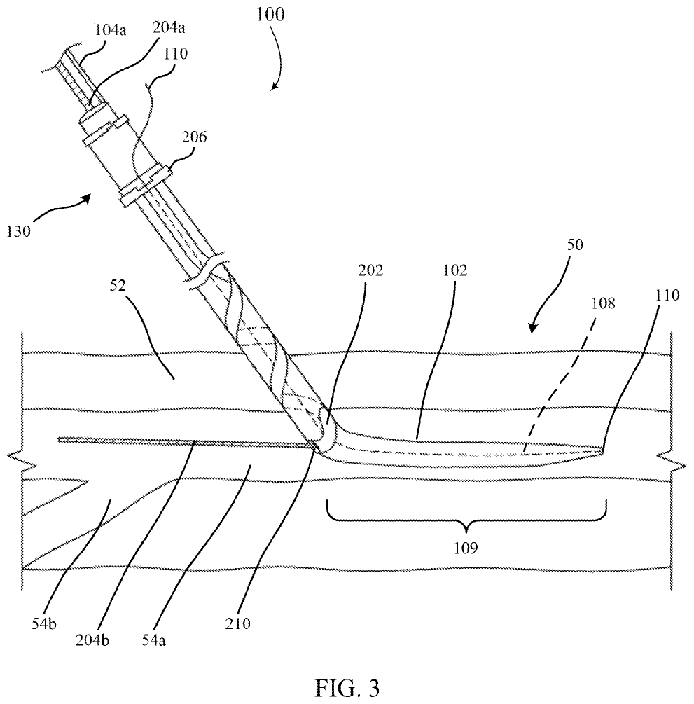

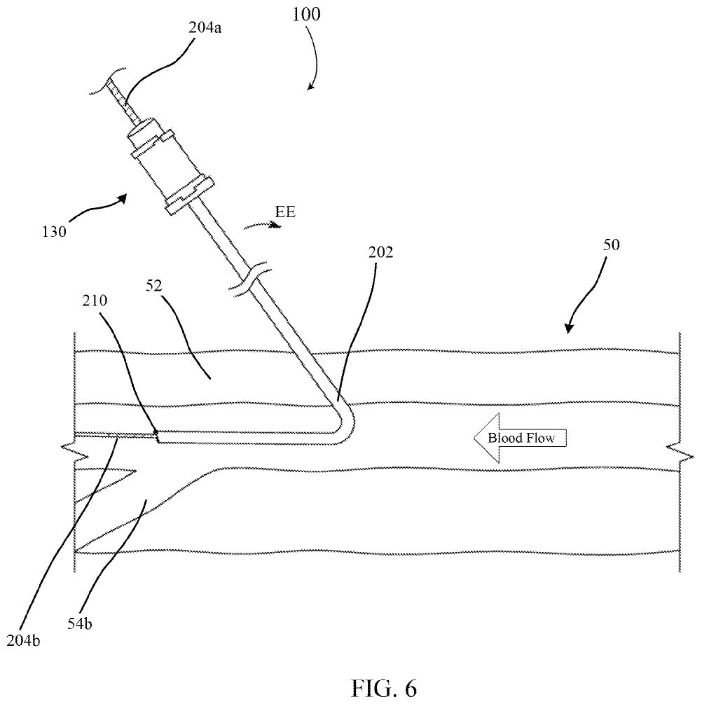

In one example, the antegrade sheath 202 may for example have stiffness that is greater than the retrograde sheath 102. In one example, the antegrade sheath 202 may for example have an elastomeric property so that the antegrade sheath 202 is configured to relax from the coiled configuration (e.g., as shown in FIGS. 1-4A) to a straight or slightly bent configuration (e.g., as shown in FIG. 6) when the retrograde sheath 102 is separated and removed. In one example, the antegrade sheath 202 may for example be formed of an elastomer that is configured to recover to a straight, substantially straight, and/or slightly bent configuration. In one example the antegrade sheath 202 may for example have stiffening braids of wire and/or composites (e.g., aramid synthetic fibers such as Kevlar®, Nomex®, and Technora®, to name a few non limiting examples). Further, the antegrade sheath 202 may for example include a radiopaque tip or radiopaque band or a plurality of bands proximal to the antegrade sheath tip. Further, the antegrade sheath 202 may for example include an atraumatic tip. For example, the tip or end portion may be tapered, chamfered or otherwise formed to prevent damage and/or minimize trauma to a patient's vasculature. The antegrade sheath 202 may for example have a hydrophilic or lubricious coating to allow for easier insertion and removal of the antegrade sheath 202.

In another aspect usable with the disclosure, the antegrade sheath 202 may for example have tip or end region that is stiffer or more rigid than a bendable portion located proximally from the tip or end region (e.g., a region shown by the bend in FIG. 6). This difference in stiffness or rigidity may allow a technician to be able to easily switch direction of the proximal end of the retrograde sheath (e.g., a technician may pivot or otherwise move the proximal end of the antegrade sheath in direction EE shown in FIG. 6). In addition, the proximal region of the antegrade sheath may have stiffness or rigidity that is greater than the bendable portion located proximally from the tip or end region and distally from the distal end of the sheath. The stiffer or more rigid proximal region to the sheath may further improve the technician's ability to easily switch direction of the proximal end of the retrograde sheath (e.g., a technician may pivot or otherwise move the proximal end of the antegrade sheath in direction EE shown in FIG. 6). In one aspect of the disclosure the center or bendable portion may have a decreased stiffness and increased flexibility and bendability with respect to the distal or tip region of the antegrade sheath and may also have a decreased stiffness and increased flexibility and bendability with respect to the proximal region of the antegrade sheath 202.

The retrograde sheath 102 may for example be configured to have a stiffness that is less than the antegrade sheath 202. In one example, the retrograde sheath 102 may for example be formed of an elastomer or other known material. In one example, the retrograde sheath 102 may for example have a connection section 108 that is configured to be separated. In one example, the connection section 108 may for example be a perforable or tear-away section that can be torn or otherwise peeled away to cause an opening, tear, or slit between the interior lumen 110 of the retrograde sheath 102 and the outer wall of the retrograde sheath 102. Further, the connection section 108 may be connected to an outer surface of the retrograde sheath 102 so that when the connection section 108 is caused to tear or release, the antegrade sheath 202 is released or otherwise separated from the retrograde sheath 102. In one example, the stiffer antegrade sheath 202 that is connected to the retrograde sheath 102 prior to separation via the connection section 108 may for example provide structural support to and provide pushablity of the sheath or catheter system 100. For example, the stiffer antegrade sheath 202 may provide structural support or pushablity to the retrograde sheath 102 allowing for the sheath or catheter system to be easily advanced over a needle or guidewire into the patients vasculature. Further, the tip region 109 (FIG. 1) may for example not have the antegrade sheath 202 connected thereto and thus may be configured to be stiffer than the remainder of the retrograde sheath 102. In one example, the tip region 109 may have a similar or slightly less stiffness than the combination of the retrograde sheath 102 with the antegrade sheath 202 connected thereto.

Once in the patient's vasculature, the retrograde sheath 102 may peeled away or otherwise separated from the antegrade sheath 202 via the connection section causing a decrease in stiffness of the retrograde sheath 102 and allowing the retrograde sheath 102 to be easily removed with the antegrade sheath 202 still within the patient's vasculature. The retrograde sheath 102 may for example include a radiopaque tip or radiopaque band or a plurality of bands proximal to the antegrade sheath tip. In one example, the tip or tip portion of the retrograde sheath 102 may have radiopaque bands or a radiopaque pattern that is different from that of the tip of the antegrade sheath 202, which allows a physician to differentiate one tip from the other and to assist a physician with verifying that the sheath system 100 is properly oriented within a patient's vasculature during a procedure. Further, the antegrade sheath 202 may for example include an atraumatic tip. For example, the tip or end portion may be tapered, chamfered or otherwise formed to prevent damage and/or minimize trauma to a patient's vasculature. The retrograde sheath 102 may for example have a hydrophilic or lubricious coating to allow for easier insertion and removal of the retrograde sheath 102. In another example, the retrograde sheath 102 may for example have stiffening braids of wire and/or composites (e.g., aramid synthetic fibers such as Kevlar®, Nomex®, and Technora®, to name a few non limiting examples). In one example implementation, the retrograde sheath 102 may have substantially the same stiffness as the antegrade sheath 202 or may have a greater stiffness than the antegrade sheath 202.

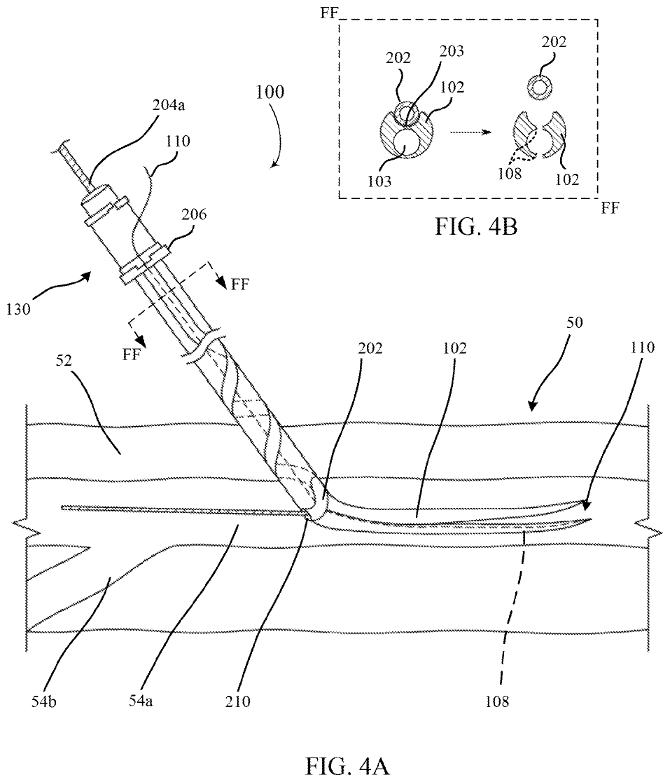

As shown in FIG. 1, when in a first configuration or retracted configuration, the antegrade sheath 202 and tip (e.g., tip near reference 210) may be configured to be flush with or substantially flush with an outer surface of the retrograde sheath 102. One example of a substantially flush configuration is shown in FIG. 4B as an example cross-section of the system 100. The retrograde sheath 102 may for example have a concave portion 203 at least partially helically provided about the axis of the retrograde sheath 102 and configured to have the antegrade sheath 202 removeably connected to and at least partially therein.

As shown in FIG. 1, the retrograde sheath 102 and antegrade sheath 202 may be connected to one another along a connection section 108 allowing for both the retrograde sheath 102 and the antegrade sheath 202 of the sheath system to be easily inserted into the vasculature (e.g., CFA 52a). As shown and as described below with respect to FIGS. 4A-6, once the system 100 is within the patients vasculature, a user of the system may pull or otherwise engage the actuator 111 causing the separation of the retrograde sheath 102 from the antegrade sheath 202 and causing the gradual relaxation of the antegrade sheath 202. In one example, the actuator 111 may for example be a wire, series of wires, and/or synthetic fiber embedded within a wall of the retrograde sheath 102 and/or the antegrade sheath 202 and/or may connect the outer wall or surface of the antegrade sheath 202 to the retrograde sheath 102. While activation is described as the pulling by a physician in the example above, it is noted that other methods of causing separation between the walls of the retrograde sheath 102 and/or the antegrade sheath 202 from the antegrade sheath are contemplated. For example, the aforementioned wire, series of sires, and/or synthetic fibers may be pulled or otherwise removed from the connection section 108 via a rotatable knob, lever or other feature that provides a tensile or pulling force causing separation of the connection section 108. In another aspect, the connection section 108 may be separated via an electronic, pneumatic and/or hydraulic actuator 111.

One example method of performing or partially performing an interventional procedure is described below with respect to FIGS. 1-6. It is noted that the method described is simplified to emphasize aspects of the disclosure, however one having ordinary skill in the art would understand that additional steps may be performed without departing from the scope of the disclosure. In certain instances and procedures, a physician may wish to access a patients legs via an access point at the CFA and may wish to access a patients legs by advancing a catheter, sheath, or other interventional device into the patients SFA. However, it generally may be preferable to perform the retrograde initial access of the CFA and redirected the sheath or catheter such that it extends down the leg in the same direction of the bloodstream (e.g., antegrade). In the past, for a physician to do this, a second incision may have been necessary in the contralateral femoral artery. Thus, a patient must be re-punctured, whereby a second tube is inserted into the femoral artery and then a long tube is directed over a wire from the contralateral femoral artery toward the ipsilateral femoral artery. However, the system 100 disclosed herein allows for a physician to reverse direction of the procedure from a retrograde direction to an antegrade direction. In one example method, a known needle may be inserted into the patients femoral artery (e.g., a patients CFA) in a retrograde direction (i.e., so that the lumen at the distal end of the needle extends against or in a reverse direction of the flow of blood in the artery. Once the needle is inserted into the artery, a guidewire be fed through the lumen of the needle to create a pathway into the femoral artery. One example of such a guidewire is shown as retrograde guidewire 104b in FIG. 1. The sheath or catheter system 100 may then be advanced over the guidewire 104b by passing the retrograde guidewire 104b through the interior lumen 110 in the retrograde sheath 102. Once the sheath system 100 is advanced over the retrograde guidewire 104b the tip region 109 of the retrograde sheath 102 may for example be facing in the retrograde direction as shown in FIG. 1. The physician may confirm the location of the tip region 109 and the tip of the antegrade sheath 202 via any one or a combination or radiopaque markings on the retrograde sheath 102 and/or the antegrade sheath 202.

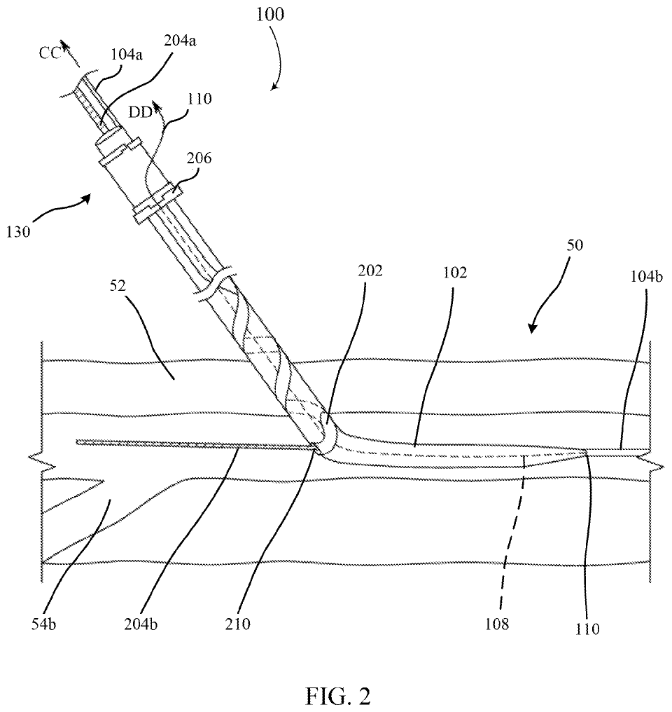

Turning now to FIG. 2, an antegrade guidewire 204a may be advanced though the lumen of the antegrade sheath 202 until the antegrade guidewire distal end 204b exits the interior lumen 210 of the antegrade sheath 202. When the antegrade sheath 202 is connected to the retrograde sheath 102, the exit of the interior lumen 210 faces in an antegrade direction allowing the antegrade guidewire 204a to be advanced in an antegrade direction that is substantially opposite the direction of the tip region 109 and/or the retrograde guidewire 104a of the retrograde sheath 102.

In one example, once it is established that the antegrade guidewire 204a is properly placed antegrade, the retrograde guidewire 104a may be removed or partially removed from the retrograde sheath 102. The activator 111 of the sheath system 100 may then be manipulated to begin separating the walls of the retrograde sheath 102 and/or the antegrade sheath 202 from the retrograde sheath 102 via the connection section 108. In one example, the activator 111 may for example be pulled by the physician as indicated by arrow DD in FIG. 2 causing the wire, series of wires, and/or synthetic fiber embedded within a wall of the retrograde sheath 102 and/or the antegrade sheath 202 and/or that may connect the outer wall or surface of the antegrade sheath 202 to the retrograde sheath 102 to be pulled from the device causing a separation in the wall of the retrograde sheath 102 and a separation between the retrograde sheath 102 and the antegrade sheath 202. While activation is described as the pulling by a physician in the example above, it is noted that other methods of causing separation between the walls of the retrograde sheath 102 and/or the antegrade sheath 202 from the antegrade sheath are contemplated. For example, the aforementioned wire, series of sires, and/or synthetic fibers may be pulled or otherwise removed from the connection section 108 via a rotatable knob, lever or other feature that provides a tensile or pulling force causing separation of the connection section 108. In another aspect, the connection section 108 may be separated via an electronic, pneumatic and/or hydraulic actuator 111. As shown in the example cross-section in FIG. 4B, engaging the activator 111 causes the separation of the retrograde sheath 102 along the connection section 108. Further, as shown in FIG. 4B, the engagement of the activator 111 causes the separation of the antegrade sheath 202 from a concave portion of the retrograde sheath 102. In one example shown in FIG. 4B, the connection section 108 may be configured to split or otherwise separate the retro into two or more halves.

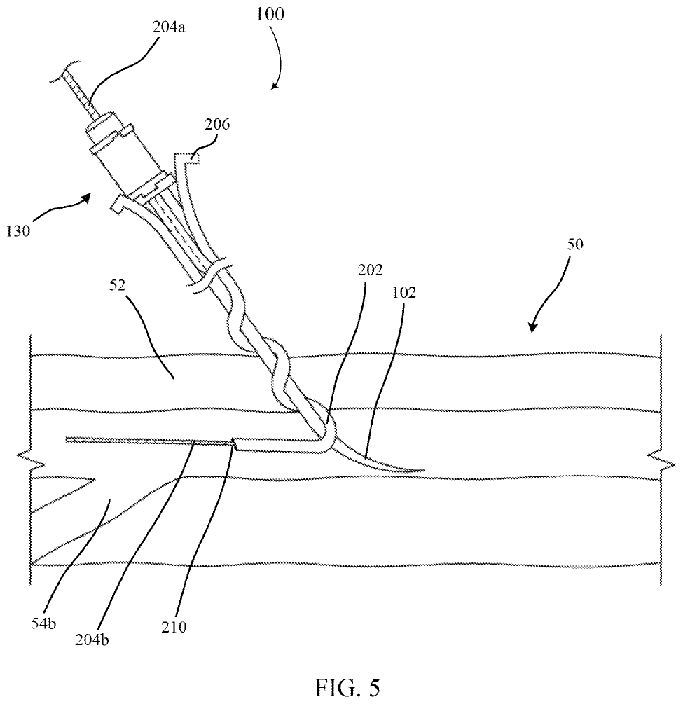

Turning now to FIG. 5, once the retrograde sheath 102 fully peeled away or separated via the connection section 108, the two or more halves of the retrograde sheath 102 may be removed from the patient. As mentioned above, in one example, the antegrade sheath 202 may relax to a naturally straight or curved state as it is no longer constrained via the connection to the retrograde sheath 102. Once the retrograde sheath 102 is fully removed the antegrade sheath 202 remains in the patient's vasculature facing in the opposite (i.e., antegrade direction) and allowing a physician to continue any interventional procedures by advancing the sheath or catheter, guidewire and/or any additional interventional devices in the same direction as the patient's blood flow (i.e., antegrade). Thus, by utilizing the aforementioned system, method, and apparatus, a physician may for example be able to conduct a procedure at a target treatment site by taking a more direct route in the patient's vasculature. For example, if the treatment site is below the knee of a patients left leg and the physician determines that antegrade initial access is not the safest or preferred entry method, the physician can now employ the aforementioned method to access the patients vasculature via retrograde entry at the left femoral artery and reverse direction of the procedure to a more direct antegrade route using the aforementioned system rather than having to enter the right femoral artery in a retrograde orientation and take a longer path to cross over to the left leg via the aortal bifurcation, for example.

FIGS. 7 -10 show another example of a catheter or sheath in accordance with one aspect of the disclosure. It is noted that the example of a catheter or sheath described with respect to FIGS. 7-10 may include similar aspects and may provide similar advantages to those described above with respect to FIGS. 1-6. Any of the methods, system, and apparatus aspects described throughout the disclosure may be used interchangeably and are not intended to be limited to one specific aspect of the disclosure. In the Example shown in FIG. 6, a sheath system 500 may be inserted in the common femoral artery (“CFA”) 354a through the tissue 352 of a patient 350. The sheath system 500 may be placed retrograde going toward the heart (i.e. against the natural direction of the bloodstream). The sheath system 500 may for example include a antegrade sheath 502 having an interior lumen 510 and an retrograde sheath 402 having an interior lumen 410. As shown in FIG. 6, the antegrade sheath 502 may be connected to the retrograde sheath 402 so as to run substantially parallel to the lumen of the retrograde sheath 402 but may be connected so as to have a curve or bend 502 (FIG. 1). As described in further detail below, the antegrade sheath 502 may be removably connected to or otherwise separable from the retrograde sheath 402. In one example aspect, the retrograde sheath 402 may for example have connection section 408. The connection section 408 may for example be a perforable or tear away section connected to a tear away activator 411. The connection section 408 may for example separate or otherwise cause a separation or break between the retrograde sheath 402 and the antegrade sheath 502 in response to an activation (e.g., a user pulling) the activator 411. Further description of the connection section 408 and activator 411 are described in further detail below. In another example, as described in further detail below, the connection section 408 may allow for separation of the antegrade sheath 502 from the retrograde sheath 402 when the retrograde sheath 402 is retracted with respect to the antegrade sheath 502 for example when the retrograde sheath 402 is retracted from a patient's vasculature by a user while holding the antegrade sheath 502 within the patients vasculature.

The sheath system may include a retrograde hub 430 with a hemostasis valve and an antegrade hub 530 with a hemostasis valve. In an alternative aspect, the hemostasis valves 430 and/or 530 may instead be an interface, such as a threaded interface that is configured to engage with (e.g., thread onto) or otherwise engage with a separate hemostasis valve (which may for example be an off-the-shelf or otherwise known hemostasis valve(s)). As an alternative, and as described in further detail above respect to FIGS. 1-6, the antegrade sheath 502 and the retrograde sheath 402 may share a single hub with corresponding first and second hemostasis valve portions. In one example, each of the hemostasis valve of hubs 430 and 530 may be configured to receive a respective one of a retrograde guidewire 404a and/or an antegrade guidewire 504a. While not shown in the figures, each one of or both of the hemostasis valves and/or hemostasis valve connection portions may for example include a sheath flush line or may be configured to connect with a sheath flush line with one or more valves and flush fitting(s) at ends thereof.

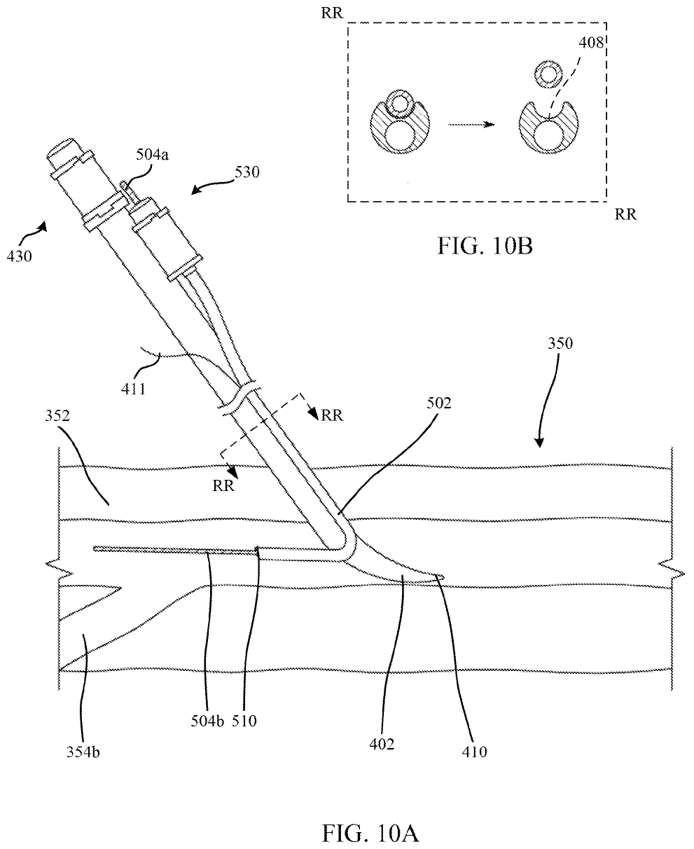

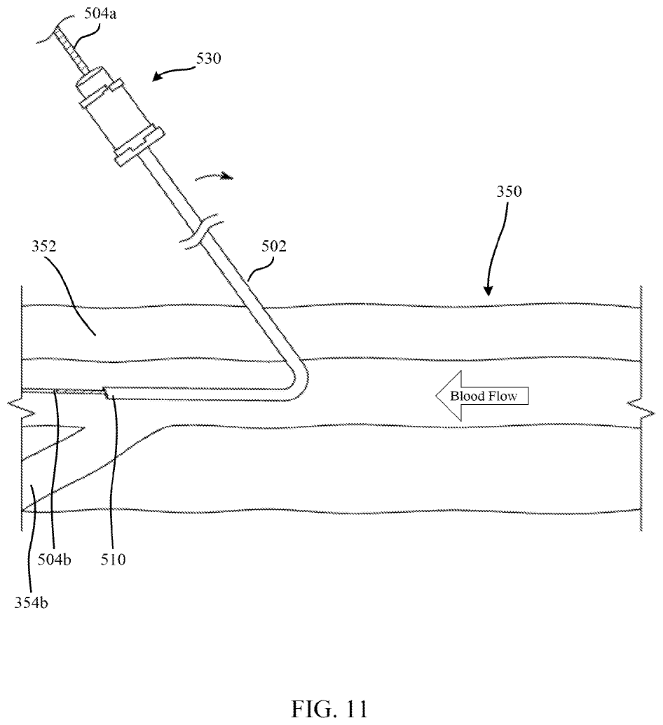

In one example, the antegrade sheath 502 may for example have stiffness that is greater than the retrograde sheath 402. In one example, the antegrade sheath 502 may for example have an elastomeric property so that the straight portion and curved portion 509 of the antegrade sheath 502 is configured to relax from the straight configuration with a curved end (e.g., as shown in FIGS. 7-9) to a slightly bent configuration (e.g., as shown in FIGS. 10A and 11) when the retrograde sheath 402 is separated and removed. In one example, the antegrade sheath 502 may for example be formed of an elastomer that is configured to recover to a straight, substantially straight, and/or slightly bent configuration. In one example the antegrade sheath 502 may for example have stiffening braids of wire and/or composites (e.g., aramid synthetic fibers such as Kevlar®, Nomex®, and Technora®, to name a few non limiting examples). Further, the antegrade sheath 502 may for example include a radiopaque tip or radiopaque band or a plurality of bands proximal to the antegrade sheath tip. Further, the antegrade sheath 502 may for example include an atraumatic tip. For example, the tip or end portion may be tapered, chamfered or otherwise formed to prevent damage and/or minimize trauma to a patient's vasculature. The antegrade sheath 502 may for example have a hydrophilic or lubricious coating to allow for easier insertion and removal of the antegrade sheath 502.

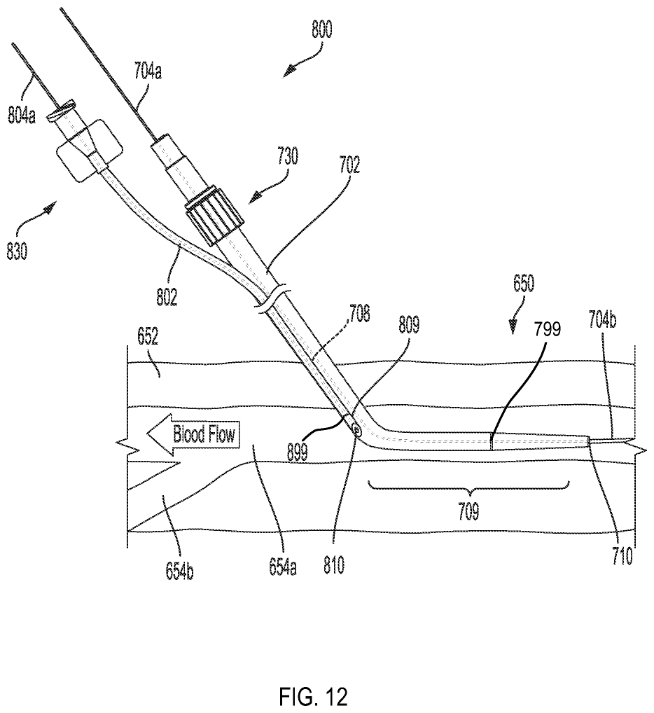

The retrograde sheath 402 may for example be configured to have a stiffness that is less than the antegrade sheath 502. In one example, the retrograde sheath 402 may for example be formed of an elastomer or other known material. In one example, the retrograde sheath 402 and/or the antegrade sheath 502 may for example have a connection section 408. In some examples, the connection section 408 may for example comprise a perforable or tear away section that is configured to be torn or otherwise peeled away to cause a tear or slit between the retrograde sheath 402 and the antegrade sheath 502 so that the antegrade sheath 502 is released or otherwise separated from the retrograde sheath 402. In some examples, the connection section may be configured to removeably contain a length of the antegrade sheath 502 as described in the example in FIGS. 12-16 below. In one example, the stiffer antegrade sheath 502 that is connected to the retrograde sheath 402 prior to separation via the connection section 408 may for example provide structural support to and provide pushablity of the sheath or catheter system 500. For example, the stiffer antegrade sheath 502 may provide structural support or pushablity to the retrograde sheath 402 allowing for the sheath or catheter system to be easily advanced over a needle or guidewire into the patient's vasculature. Further, the tip region 409 (FIG. 7) may for example not have the antegrade sheath 502 connected thereto and thus may be configured to be stiffer than the remainder of the retrograde sheath 402. In one example, the tip region 409 may have a similar or slightly less stiffness than the combination of the retrograde sheath 402 with the antegrade sheath 502 connected thereto.

Once in the patient's vasculature, the retrograde sheath 402 may peeled away or otherwise separated from the antegrade sheath 502 via the connection section perforable or tear away section causing a decrease in stiffness of the retrograde sheath 402 and allowing the retrograde sheath 402 to be easily removed with the antegrade sheath 502 still within the patient's vasculature. The retrograde sheath 402 may for example include a radiopaque tip or radiopaque band or a plurality of bands proximal to the antegrade sheath tip. In one example, the tip or tip portion of the retrograde sheath 402 may have radiopaque bands or a radiopaque pattern that is different from that of the tip of the antegrade sheath 502, which allows a physician to differentiate one tip from the other and to assist a physician with verifying that the sheath system 500 is properly oriented within a patient's vasculature during a procedure. Further, the antegrade sheath 502 may for example include an atraumatic tip. For example, the tip or end portion may be tapered, chamfered or otherwise formed to prevent damage and/or minimize trauma to a patient's vasculature. The retrograde sheath 402 may for example have a hydrophilic or lubricious coating to allow for easier insertion and removal of the retrograde sheath 402. In another example, the retrograde sheath 402 may for example have stiffening braids of wire and/or composites (e.g., aramid synthetic fibers such as Kevlar®, Nomex®, and Technora®, to name a few non limiting examples). In one example implementation, the retrograde sheath 402 may have substantially the same stiffness as the antegrade sheath 502 or may have a greater stiffness than the antegrade sheath 502.

In another aspect usable with the disclosure, the antegrade sheath 502 may for example have tip or end region that is stiffer or more rigid than a bendable portion located proximally from the tip or end region (e.g., a region shown by the bend in FIG. 11). This difference in stiffness or rigidity may allow a technician to be able to easily switch direction of the proximal end of the retrograde sheath (e.g., a technician may pivot or otherwise move the proximal end of the antegrade sheath in direction EE shown in FIG. 6 or as indicated by the arrow in FIG. 11). In addition, the proximal region of the antegrade sheath may have stiffness or rigidity that is greater than the bendable portion located proximally from the tip or end region and distally from the distal end of the sheath. The stiffer or more rigid proximal region to the sheath may further improve the technician's ability to easily switch direction of the proximal end of the retrograde sheath (e.g., a technician may pivot or otherwise move the proximal end of the antegrade sheath in direction EE shown in FIG. 6 or as indicated by the arrow in FIG. 11). In one aspect of the disclosure the center or bendable portion may have a decreased stiffness and increased flexibility and bendability with respect to the distal or tip region of the antegrade sheath and may also have a decreased stiffness and increased flexibility and bendability with respect to the proximal region of the antegrade sheath 502.

As shown in FIG. 7, when in a first configuration or retracted configuration, the antegrade sheath 502 and tip (e.g., tip near reference 510) may be configured to be flush with or substantially flush with an outer surface of the retrograde sheath 402. One example of a substantially flush configuration is shown in FIG. 10B as an example cross-section of the system 500. The retrograde sheath 402 may for example have a concave portion 408 at least partially running parallel to the the axis of the retrograde sheath 402 and configured to have the antegrade sheath 502 removeably connected to and at least partially contained therein.

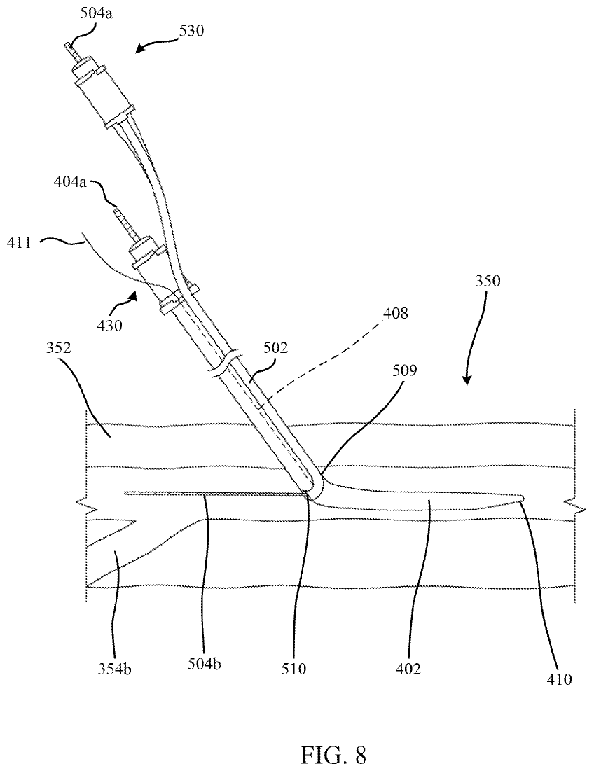

As shown in FIG. 7, the retrograde sheath 402 and antegrade sheath 502 may be connected to one another along a connection section 408 allowing for both the retrograde sheath 402 and the antegrade sheath 502 of the sheath system to be easily inserted into the vasculature (e.g., CFA 354a). As shown and as described below with respect to FIGS. 8-11, once the system 500 is within the patient's vasculature, a user of the system may pull or otherwise engage the actuator 511 causing the separation of the retrograde sheath 402 from the antegrade sheath 502 and causing the gradual relaxation of the antegrade sheath 502. In another example, the user of the system may retract the retrograde sheath 402 with respect to the antegrade sheath 502 causing the separation of the retrograde sheath 402 from the antegrade sheath and causing the gradual relaxation of the antegrade sheath 502. In the aforementioned example utilizing the actuator 511, the actuator 511 may for example be a wire, series of wires, and/or synthetic fiber embedded within a wall of the retrograde sheath 402 and/or the antegrade sheath 502 and/or may connect the outer wall or surface of the antegrade sheath 502 to the retrograde sheath 402. While activation is described as the pulling by a physician in the example above, it is noted that other methods of causing separation between the walls of the retrograde sheath 402 and/or the antegrade sheath 502 from the antegrade sheath are contemplated. For example, the aforementioned wire, series of sires, and/or synthetic fibers may be pulled or otherwise removed from the connection section 408 via a rotatable knob, lever or other feature that provides a tensile or pulling force causing separation of the connection section 408. In another aspect, the connection section 408 may be separated via an electronic, pneumatic and/or hydraulic actuator 411.

One example method of performing or partially performing an interventional procedure is described below with respect to FIGS. 7-10. As mentioned in the examples above, above the method described is simplified to emphasize aspects of the disclosure, however one having ordinary skill in the art would understand that additional steps may be performed without departing from the scope of the disclosure. Similarly, to the aspects described above with respect to FIGS. 1-6, the system 500 disclosed described with respect to FIGS. 7-11 allows for a physician to reverse direction of the procedure from a retrograde direction to an antegrade direction.

In one example method, a known needle may be inserted into the patient's femoral artery (e.g., a patients CFA) in a retrograde direction (i.e., so that the lumen at the distal end of the needle extends against or in a reverse direction of the flow of blood in the artery. Once the needle is inserted into the artery, a guidewire be fed through the lumen of the needle to create a pathway into the femoral artery. One example of such a guidewire is shown as retrograde guidewire with a proximal end 404a and a distal end 404b in FIG. 1. The sheath or catheter system 500 may then be advanced over the guidewire (404a, 404b) by passing the retrograde guidewire (404a, 404b) through the interior lumen 410 in the retrograde sheath 402. Once the sheath system 500 is advanced over the retrograde guidewire (404a, 404b) the tip region 409 of the retrograde sheath 402 may for example be facing in the retrograde direction as shown in FIG. 7. The physician may confirm the location of the tip region 409 and the tip of the antegrade sheath (e.g., near reference 510) via any one or a combination or radiopaque markings on the retrograde sheath 402 and/or the antegrade sheath 502.

Turning now to FIG. 8, an antegrade guidewire with a proximal end 504a and a distal end 504b may be advanced though the lumen of the antegrade sheath 502 until the antegrade guidewire distal end 504b exits the interior lumen 510 of the antegrade sheath 502. When the antegrade sheath 502 is connected to the retrograde sheath 402, the exit of the interior lumen 510 faces in an antegrade direction allowing the antegrade guidewire 504b to be advanced in an antegrade direction that is substantially opposite the direction of the tip region 409 and/or the retrograde guidewire 404b of the retrograde sheath 102.

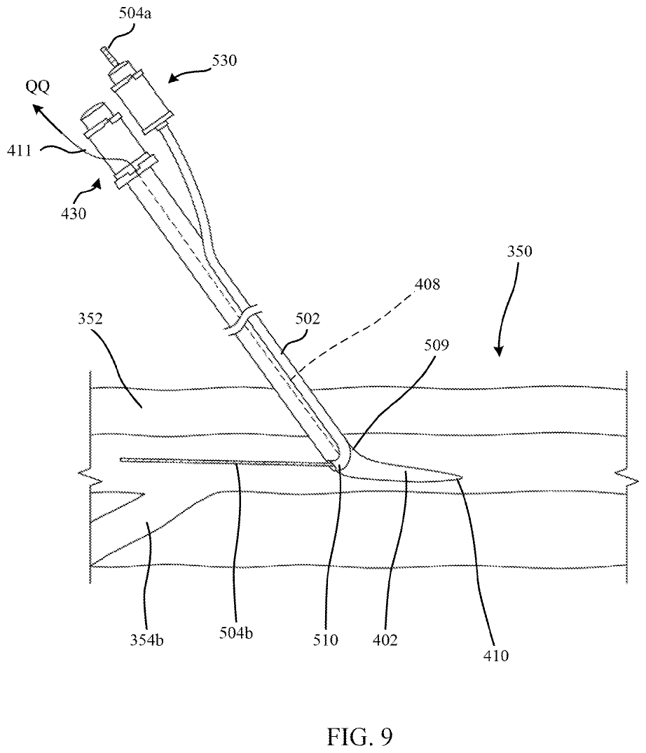

In one example, once it is established that the distal end of the antegrade guidewire 504b is properly placed antegrade, the retrograde guidewire (404a, 404b) may be removed or partially removed from the retrograde sheath 402. The activator 411 of the sheath system 500 may then be manipulated to begin separating the walls of the retrograde sheath 402 and/or the antegrade sheath 502 from one another via the connection section 408. In one example, the activator 411 may for example be pulled by the physician as indicated by arrow QQ in FIG. 9 causing the wire, series of wires, and/or synthetic fiber embedded within a wall of the retrograde sheath 402 and/or the antegrade sheath 502 to shear the connection connect between the outer wall or surface of the antegrade sheath 502 to the retrograde sheath 402. While activation is described as the pulling by a physician in the example above, it is noted that other methods of causing separation between the walls of the retrograde sheath 402 and/or the antegrade sheath 502 from the antegrade sheath are contemplated. For example, the aforementioned wire, series of sires, and/or synthetic fibers may be pulled or otherwise removed from the connection section 408 via a rotatable knob, lever or other feature that provides a tensile or pulling force causing separation of the connection section 408. In another aspect, the connection section 408 may be separated via an electronic, pneumatic and/or hydraulic actuator 411. As shown in the example cross-section in FIG. 10B, engaging the activator 411 causes the separation of the antegrade sheath 502 from a concave portion of the retrograde sheath 402.

Turning now to FIGS. 10A and 11, once the retrograde sheath 402 fully separated via the connection section 408 from the antegrade sheath 502, the retrograde sheath 402 may be removed from the patient. As mentioned above, in one example, the antegrade sheath 502 may relax to a naturally straight or curved state as shown in FIGS. 10A and 11 as it is no longer constrained via the connection to the retrograde sheath 402. Once the retrograde sheath 402 is fully removed the antegrade sheath 502 remains in the patient's vasculature facing in the opposite (i.e., antegrade direction) and allowing a physician to continue any interventional procedures by advancing the sheath or catheter, guidewire and/or any additional interventional devices in the same direction as the patient's blood flow (i.e., antegrade). Similarly, to the advantages above, one example advantage of the aforementioned disclosure is that by utilizing the aforementioned system, method, and apparatus, a physician may for example be able to conduct a procedure at a target treatment site by taking a more direct route in the patient's vasculature. For example, if the treatment site is below the knee of a patients left leg and the physician determines that antegrade initial access is not the safest or preferred entry method, the physician can now employ the aforementioned method to access the patients vasculature via retrograde entry at the left femoral artery and reverse direction of the procedure to a more direct antegrade route using the aforementioned system rather than having to enter the right femoral artery in a retrograde orientation and take a longer path to cross over to the left leg via the aortal bifurcation, for example

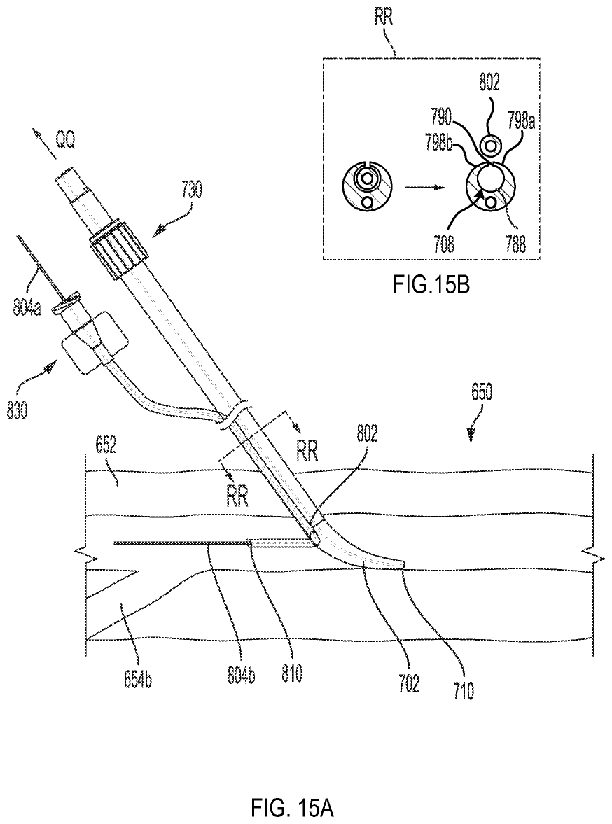

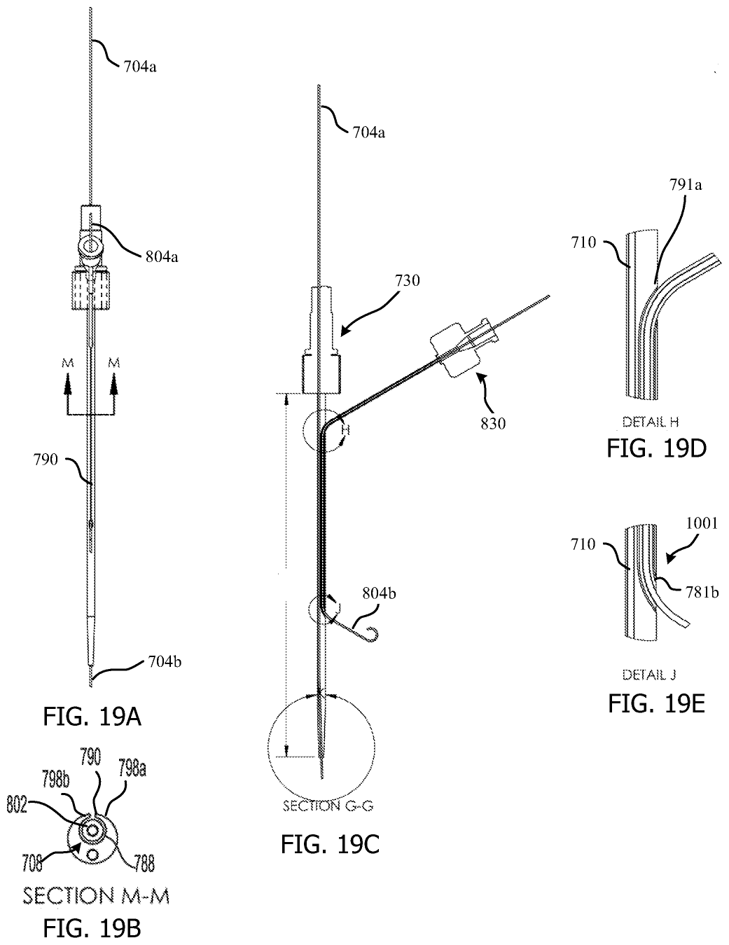

FIGS. 12-16 show another example of a catheter or sheath in accordance with one aspect of the disclosure. It is noted that the example of a catheter or sheath described with respect to FIGS. 12-16 may interchangeably include any one or combination of aspects of or may be analogous with the catheter or sheath described above with respect to FIGS. 1-11 and may provide similar advantages. Any of the methods, system, and apparatus aspects described throughout the disclosure may be used interchangeably and are not intended to be limited to one specific aspect of the disclosure. In the Example shown in FIG. 12, a sheath system 800 may be inserted in the common femoral artery (“CFA”) 654a through the tissue 652 of a patient 650. The sheath system 800 may be placed retrograde going toward the heart (i.e., against the natural direction of the bloodstream). The sheath system 800 may for example include an antegrade sheath 802 having an interior lumen 810 and a retrograde sheath 702 having an interior lumen 710. As shown in FIG. 12, the antegrade sheath 802 may be removeably connected to the retrograde sheath 702 so as to run substantially parallel to the lumen 710 of the retrograde sheath 702 but may be connected so as to have a curve or bend 1001 (shown in FIG. 19E). As described in further detail below, the antegrade sheath 802 may be removably connected to or otherwise separable from the retrograde sheath 702. In one example aspect, the retrograde sheath 702 may for example have connection section 708. The connection section 708 may for example be a perforable or tear away section as described above and/or may have a concave section or trough. In a preferred aspect, the connection section 708 may have an axially extending concave trough or channel 788 and a first and second wall 798a and 789b, respectively (see FIGS. 15B, 17B, 18A-18F, and 19A-19E). The first wall 798a and second wall 798b may for example be separated via an axially extending aperture or slit 790. The first wall 798a, second wall 798b, and channel 788 may be configured to captively retain a length of the antegrade sheath 802. However, when a user retracts or otherwise pulls away the retrograde sheath 702 from the antegrade sheath 802, the first wall 798a and the second wall 798b may for example separate to allow the antegrade sheath 802 to partially separate or fully separate from the retrograde sheath 702. As described in further detail below, the connection section 708 may allow for separation of the antegrade sheath 802 from the retrograde sheath 702 when the retrograde sheath 702 is retracted with respect to the antegrade sheath 802 when the retrograde sheath 702 is retracted from a patient's vasculature by a user while holding the antegrade sheath 802 within the patient's vasculature. Additional details and aspects of the connection section 708 are described in further detail below.

In another example, the connection section connection section 708 may for example separate or otherwise cause a separation or break between the retrograde sheath 702 and the antegrade sheath 802 in response to an activation (e.g., a user pulling an activator and/or twisting the antegrade sheath 802 with respect to the retrograde sheath 702).

The sheath system 800 may include a retrograde hub 730 with a hemostasis valve and an antegrade hub 830 with a hemostasis valve. In an alternative aspect, any one of or both of the hemostasis valves 730 and/or 830 may instead be an interface, such as a threaded interface (e.g., a Luer fitting) that is configured to engage with (e.g., thread onto) or otherwise engage with a separate hemostasis valve (which may for example be an off-the-shelf or otherwise known hemostasis valve(s)). As an alternative, and as described in further detail above respect to FIGS. 1-6, the antegrade sheath 802 and the retrograde sheath 702 may share a single hub with corresponding first and second hemostasis valve portions. In one example, each of the hemostasis valve of hubs 730 and 830 may be configured to receive a respective one of a retrograde guidewire 704a and/or an antegrade guidewire 804a. While not shown in the figures, each one of or both of the hemostasis valves and/or hemostasis valve connection portions 730 and/or 830 may for example include a sheath flush line or may be configured to connect with a sheath flush line with one or more valves and flush fitting(s) at ends thereof.

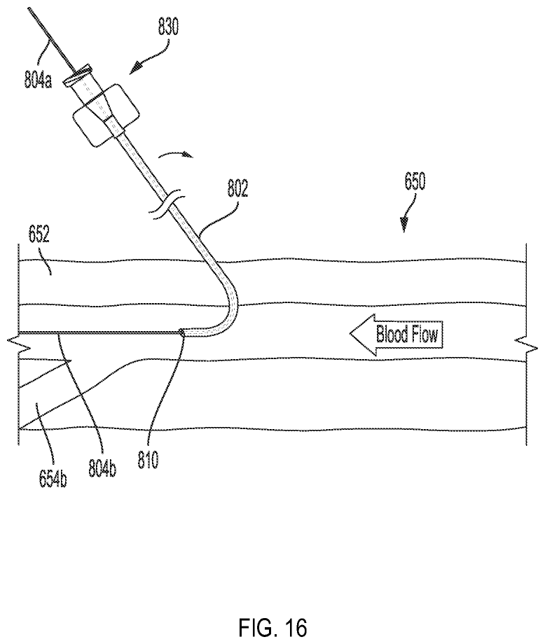

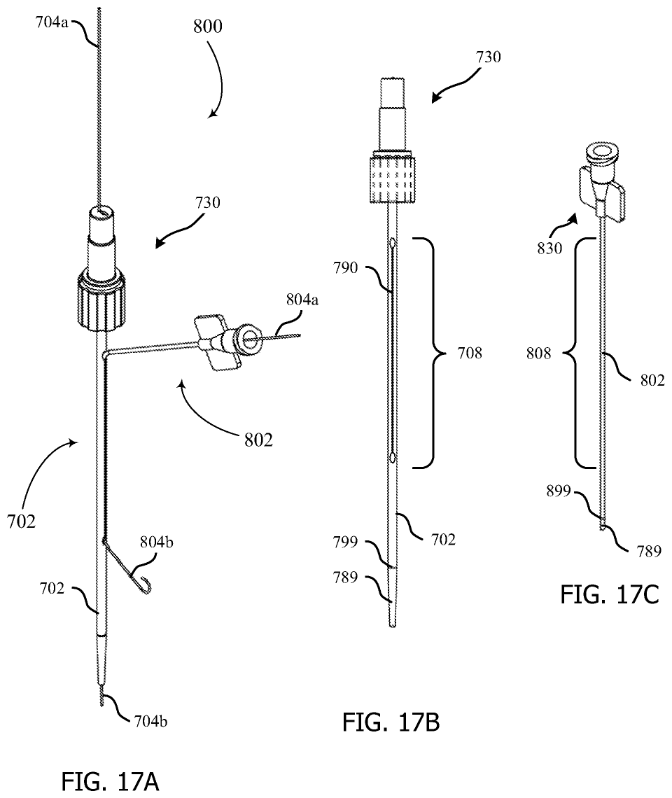

In one example, the antegrade sheath 802 may for example have stiffness that is greater than the retrograde sheath 702. Further, the antegrade sheath 802 may have an elastomeric property so that the straight portion and curved portion 809 of the antegrade sheath 802 is configured to relax from the straight configuration with a curved end (e.g., as see curved portion 509 as shown in FIGS. 7-9) to a slightly bent configuration (e.g., as shown in FIGS. 11 and 16) when the retrograde sheath 702 is separated, retracted, and removed. In one example, the antegrade sheath 802 may for example be formed of an elastomer that is configured to recover to a straight, substantially straight, and/or slightly bent configuration (e.g., as shown in FIG. 16). In one example, the antegrade sheath 802 may for example have stiffening braids of wire and/or composites (e.g., aramid synthetic fibers such as Kevlar®, Nomex®, and Technora®, to name a few non limiting examples). Further, the antegrade sheath 802 and/or retrograde sheath 702 may for example include a radiopaque tip or radiopaque band or a plurality of bands e.g., radiopaque band or markers 799 and/or 899 (FIGS. 12, 17B, and 17C) proximal to the retrograde and/or antegrade sheath tip. Further, either one of or both of the antegrade sheath 802 and/or retrograde sheath 702 may for example include an atraumatic tip 889 (FIG. 17C), and 789 (FIG. 17B). For example, the tip or end portion may be tapered, chamfered or otherwise formed to prevent damage and/or minimize trauma to a patient's vasculature. The antegrade sheath 802 and/or retrograde sheath 702 may for example have a hydrophilic or lubricious coating to allow for easier insertion and removal of the sheath.

The retrograde sheath 702 may for example be configured to have a stiffness that is less than the antegrade sheath 802. In one example, the retrograde sheath 702 may for example be formed of an elastomer or other known material. In one example, the retrograde sheath 702 and/or the antegrade sheath 802 may for example have a connection section 708 and connection section 808 (FIG. 17C), respectively. In some examples, the connection section 708 may for example comprise a connection section 708 configured to captively contain the connection section 808 of the antegrade sheath 802. The connection section 708 may have a first wall 798a and/or a second wall 798b with a slit 790 therebetween. The first wall 798a and/or the second wall 798b may be flexible or have an elastomeric property that causes the first wall 798a and/or the second wall 798b to form an axially extending trough or lumen that is configured to contain a section of the antegrade sheath 802, while allowing the first wall 798a and/or second wall 798b to flex causing an increase in a dimension of the slit 790, thus causing selective release of the antegrade sheath 802 from the connection section 708 for example when a user retracts the retrograde sheath 702 from the antegrade sheath 802 and/or from a patients vasculature. For example, such expansion of the slit 790 and separation of the antegrade sheath 802 from the retrograde sheath 702 may occur if a user holds the antegrade sheath 802 substantially stationary while pulling the retrograde sheath 702 distally away from the antegrade sheath 802. In one example, the stiffer antegrade sheath 802 that is connected to the retrograde sheath 702 prior to separation via the connection section 708 may for example provide structural support to and provide pushablity of the sheath or catheter system 800. For example, the stiffer antegrade sheath 802 may provide structural support or pushablity to the retrograde sheath 702 allowing for the sheath or catheter system to be easily advanced over a needle or guidewire into the patient's vasculature. Further, the tip region 709 (FIG. 12) may for example not have the antegrade sheath 802 connected thereto and thus may be configured to be stiffer than the remainder of the retrograde sheath 702. In one example, the tip region 709 may have a similar or slightly less stiffness than the combination of the retrograde sheath 702 with the antegrade sheath 802 connected thereto.

Once in the patient's vasculature, the retrograde sheath 702 may peeled away or otherwise separated from the antegrade sheath 802 via the connection section 708 causing a decrease in stiffness of the retrograde sheath 702 and allowing the retrograde sheath 702 to be easily removed with the antegrade sheath 802 still within the patient's vasculature. The retrograde sheath 702 may for example include a radiopaque tip or radiopaque band or a plurality of bands 799 (FIGS. 12 and 17B) proximal to the antegrade sheath tip. In one example, the tip or tip portion of the retrograde sheath 702 may have radiopaque bands 899 (FIGS. 12 and 17C) or a radiopaque pattern that is different from that of the tip of the antegrade sheath 802, which allows a physician to differentiate one tip from the other and to assist a physician with verifying that the sheath system 800 is properly oriented within a patient's vasculature during a procedure. The retrograde sheath 702 may for example have a hydrophilic or lubricious coating to allow for easier insertion and removal of the retrograde sheath 702. In another example, the retrograde sheath 402 may for example have stiffening braids of wire and/or composites (e.g., aramid synthetic fibers such as Kevlar®, Nomex®, and Technora®, to name a few non limiting examples). In one example implementation, the retrograde sheath 702 may have substantially the same stiffness as the antegrade sheath 802 or may have a greater stiffness than the antegrade sheath 802.

In another aspect usable with the disclosure, the antegrade sheath 802 may for example have tip or end region that is stiffer or more rigid than a bendable portion located proximally from the tip or end region (e.g., a region shown by the bend in FIG. 16). This difference in stiffness or rigidity may allow a technician to be able to easily switch direction of the proximal end of the retrograde sheath (e.g., a technician may pivot or otherwise move the proximal end of the antegrade sheath in direction EE shown in FIG. 6 or as indicated by the arrow in FIG. 11). In addition, the proximal region of the antegrade sheath may have stiffness or rigidity that is greater than the bendable portion located proximally from the tip or end region and distally from the distal end of the sheath. The stiffer or more rigid proximal region to the sheath may further improve the technician's ability to easily switch direction of the proximal end of the retrograde sheath (e.g., a technician may pivot or otherwise move the proximal end of the antegrade sheath in direction EE shown in FIG. 6 or as indicated by the arrow in FIG. 16). In one aspect of the disclosure the center or bendable portion may have a decreased stiffness and increased flexibility and bendability with respect to the distal or tip region of the antegrade sheath and may also have a decreased stiffness and increased flexibility and bendability with respect to the proximal region of the antegrade sheath 802.

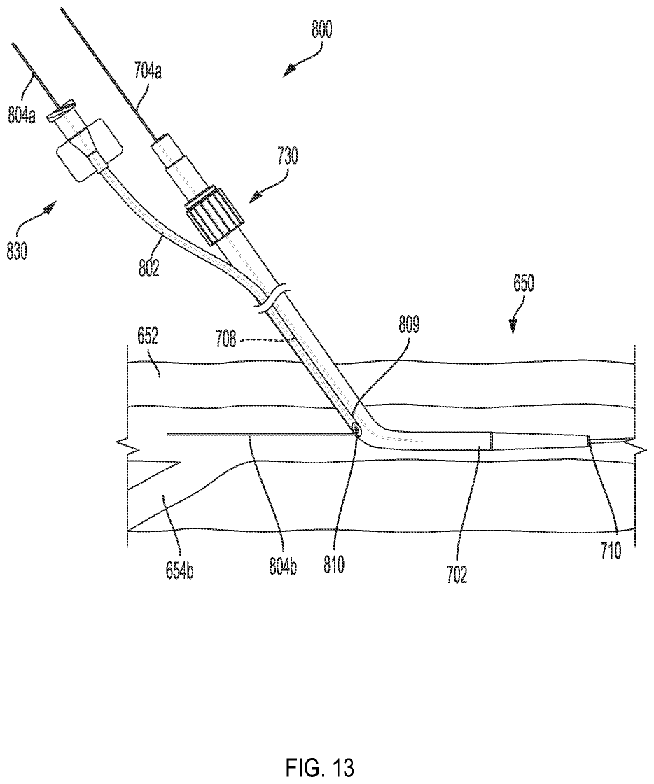

As shown in FIG. 13, when in a first configuration or retracted configuration, the antegrade sheath 802 and tip (e.g., tip near reference 810) may be configured to be flush with or substantially flush with an outer surface of the retrograde sheath 702. One example of a substantially flush configuration is shown in FIG. 15B as an example cross-section of the system 800. The retrograde sheath 702 may for example have a concave portion 788 at least partially running parallel to the axis of the retrograde sheath 702 and configured to have the antegrade sheath 802 removeably connected to and at least partially contained therein via a first wall 798a and/or a second wall 798b.

As shown in FIG. 12, the retrograde sheath 702 and antegrade sheath 802 may be removeably connected to one another along a connection section 708 allowing for both the retrograde sheath 702 and the antegrade sheath 802 of the sheath system to be easily inserted into the vasculature (e.g., CFA 654a). As shown and as described below with respect to FIGS. 12-16, once the system 800 is within the patient's vasculature, a user of the system 800 may retract the retrograde sheath 702 from the antegrade sheath 802 causing the separation of the antegrade sheath 802 from the retrograde sheath 702. The aforementioned separation may cause the gradual relaxation of the antegrade sheath 802. While activation is described as the retraction of the retrograde sheath 702 form the antegrade sheath 802 by a physician in the example above, it is noted that other methods of causing separation between the walls of the retrograde sheath 702 and/or the antegrade sheath 802 from the antegrade sheath are contemplated. For example, methods described above with respect to FIGS. 1-11 utilizing a wire, series of wires, and/or synthetic fibers may be pulled or otherwise removed from the connection section 708 via a rotatable knob, lever or other feature that provides a tensile or pulling force causing separation of the connection section 708. In another aspect, the connection section 708 may be separated via an electronic, pneumatic and/or hydraulic actuator.

One example method of performing or partially performing an interventional procedure is described below with respect to FIGS. 12-16. As mentioned in the examples above, above the method described is simplified to emphasize aspects of the disclosure, however one having ordinary skill in the art would understand that additional steps may be performed without departing from the scope of the disclosure. Further, the steps described below may share similarities with or may be analogous with the steps described above with respect to FIGS. 1-11. The system 800 described herein used with the disclosed device and system features described allows for a physician to reverse direction of the procedure from a retrograde direction to an antegrade direction.

In one example method, a known needle may be inserted into the patient's femoral artery (e.g., a patients CFA 654a) in a retrograde direction (i.e., so that the lumen at the distal end of the needle extends against or in a reverse direction of the flow of blood in the artery. Once the needle is inserted into the artery, a guidewire may be fed through the lumen of the needle to create a pathway into the femoral artery. One example of such a guidewire is shown as retrograde guidewire with a proximal end 704a and a distal end 704b in FIG. 12. The sheath or catheter system 800 may then be advanced over the guidewire (704a, 704b) by passing the retrograde guidewire (704a, 704b) through the interior lumen 710 in the retrograde sheath 702. Once the sheath system 800 is advanced over the retrograde guidewire (704a, 704b) the tip region 709 of the retrograde sheath 702 may be facing in the retrograde direction as shown in FIG. 12. The physician may confirm the location of the tip region 709 and the tip of the antegrade sheath 802 via any one or a combination of radiopaque markings 799 on the retrograde sheath 702 and/or via any one or a combination of radiopaque markings 899 on the antegrade sheath 802. In one example, usable in combination with or as an alternative to the steps described above, a physician may verify location of the tip region 709 of the retrograde sheath 702 in the CFA 654a by verifying blood flow from the distal end of the retrograde sheath 702 at the retrograde hub 730.

Turning now to FIG. 13, an antegrade guidewire with a proximal end 804a and a distal end 804b may be advanced though the lumen of the antegrade sheath 802 until the antegrade guidewire distal end 804b exits the interior lumen 810 of the antegrade sheath 802. When the antegrade sheath 502 is connected to the retrograde sheath 702, the exit of the interior lumen 810 faces in an antegrade direction allowing the distal end of the antegrade guidewire 804b to be advanced in an antegrade direction that is substantially opposite the direction of the tip region 709 (FIG. 12) and/or the retrograde guidewire 704b of the retrograde sheath 702. The physician may confirm the location of the tip region (e.g., tip near reference number 810) of the antegrade sheath 802 via any one or a combination of radiopaque markings 899 on the antegrade sheath 802. In one example usable in combination with or as an alternative to the steps described above, a physician may verify location of the tip region of the antegrade sheath 802 in the CFA 654a by verifying blood flow from the distal end of the antegrade sheath 802 at the antegrade hub 830.

In one example method, once it is established that the distal end of the antegrade guidewire 804b is properly placed antegrade, the retrograde guidewire (704a, 704b) may be removed or partially removed from the retrograde sheath 702. In another example, the retrograde guidewire (704a, 704b) may remain in the retrograde sheath 702 as the retrograde sheath is withdrawn as described in further detail below.

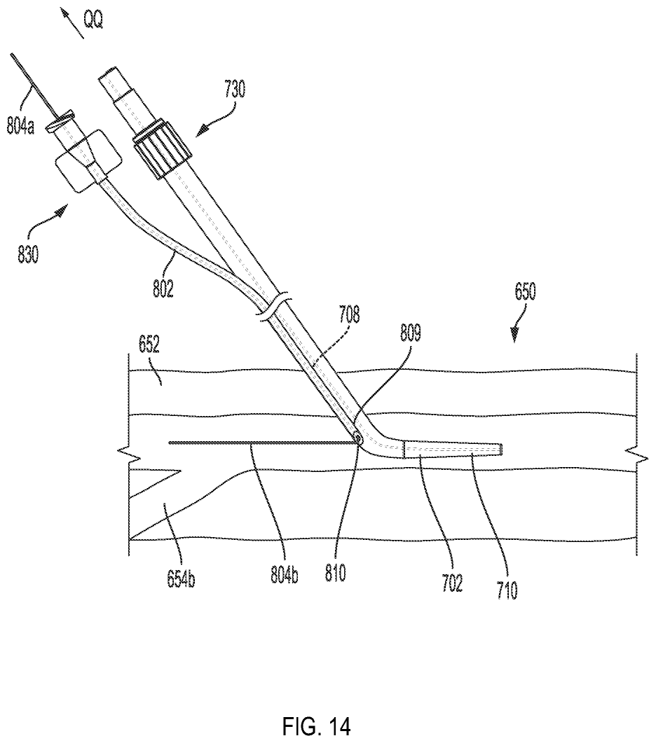

The antegrade sheath 802 and/or the retrograde sheath 702 may then be manipulated to begin separating the retrograde sheath 702 from the antegrade sheath 802 via the connection section 708. In one example, the antegrade sheath 802 may (e.g., the antegrade hub 830) may be held relatively stationary as the retrograde sheath 702 is pulled (e.g., via the retrograde hub 730) by the physician as indicated by arrow QQ in FIGS. 14 and 15A. The aforementioned manipulation may cause the first wall 798a (FIG. 15B) and/or second wall 798b to separate at the slit 790 thus releasing the antegrade sheath 802 from the retrograde sheath 702 as the retrograde sheath 702 is removed from the CFA 654a (FIG. 12). As shown in the example cross-section in FIG. 15B, withdrawing the retrograde sheath 702 with respect to the antegrade sheath 802 may cause separation of the connection section 808 of the antegrade sheath from the connection section 708 of the retrograde sheath. As mentioned above, the aforementioned connection section 708 may instead include a tear away or separable section and may include any one or combination of a wires, series of sires, and/or synthetic fibers may be pulled or otherwise removed from the connection section 708 via a rotatable knob, lever or other feature that provides a tensile or pulling force causing separation of the connection section 408. In another aspect, the connection section 708 may be separated via an electronic, pneumatic and/or hydraulic actuator.

Turning now to FIGS. 15A-B and 16, once the retrograde sheath 702 is fully separated via the connection section 708 from the antegrade sheath 802, the retrograde sheath 702 may be removed from the patient. As mentioned above, in one example, the antegrade sheath 802 may relax to a naturally straight or curved state as shown in FIGS. 115A and 16 as it is no longer constrained via the connection to the retrograde sheath 702. Once the retrograde sheath 702 is fully removed the antegrade sheath 802 remains in the patient's vasculature facing in the opposite (i.e., antegrade direction) and allows a physician to continue any interventional procedures by advancing the sheath or catheter, guidewire and/or any additional interventional devices in the same direction as the patient's blood flow (i.e., antegrade). In one example, the physician may for example withdraw the antegrade sheath 802, and utilize the antegrade guidewire 804a, 804b to advance a catheter or sheath. In another example, the physician may utilize the antegrade sheath 802 to advance any one or combination of interventional devices. In yet another example, the physician may exchange the antegrade guidewire for a different guidewire (i.e., differing in any one or a combination of length, thickness and/or shape) and may then proceed in an interventional procedure via the guidewire.

Similarly, to the advantages above, one example advantage of the aforementioned disclosure is that by utilizing the aforementioned system, method, and apparatus, a physician may for example be able to conduct a procedure at a target treatment site by taking a more direct route in the patient's vasculature. For example, if the treatment site is below the knee of a patients left leg and the physician determines that antegrade initial access is not the safest or preferred entry method, the physician can now employ the aforementioned method to access the patients vasculature via retrograde entry at the left femoral artery and reverse direction of the procedure to a more direct antegrade route using the aforementioned system rather than having to enter the right femoral artery in a retrograde orientation and take a longer path to cross over to the left leg via the aortal bifurcation, for example

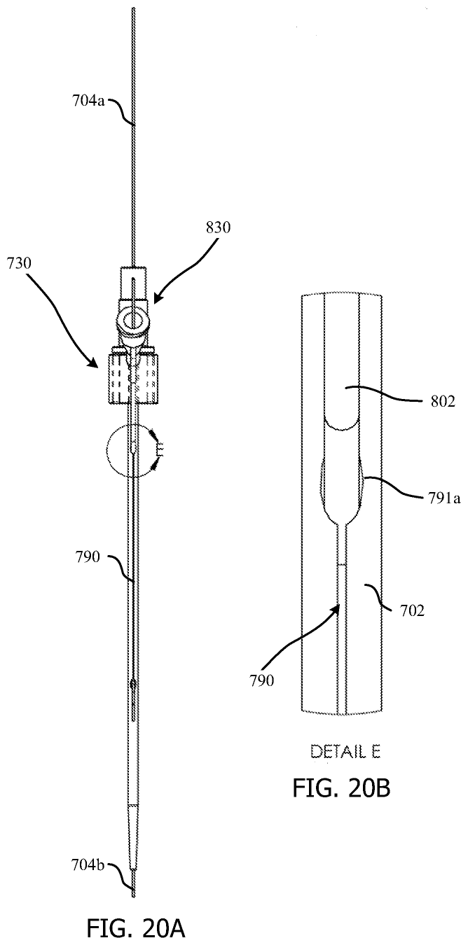

FIGS. 17A-20B show examples of various features of the device, system, and method(s) described above. For example, FIGS. 17A, 19A-20B show one example of a sheath system 800 according to aspects of the disclosure with a connection section 808 of the antegrade sheath 802 engaged with and otherwise contained within the connection section 708 of the retrograde sheath 702. The sheath system 800 shown may be analogous with the sheath system 100 in FIGS. 1-6, the sheath system 500 in FIGS. 7-11 and/or the sheath system 800 in FIGS. 12-16.

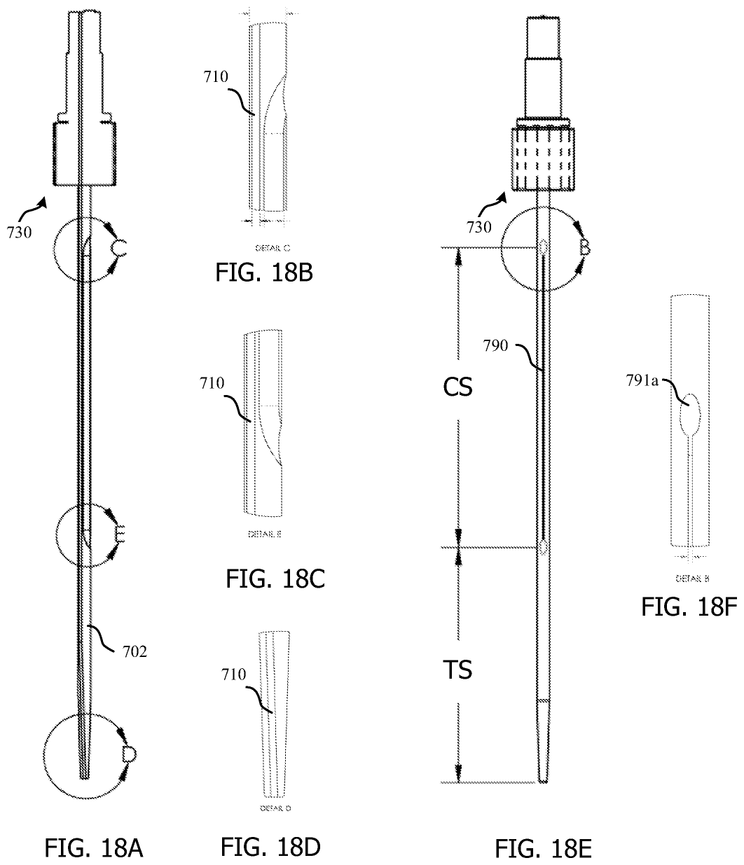

FIGS. 17B, and 18A-F show example views and features of a retrograde sheath 702 as described above. The retrograde sheath 702 may for example be analogous with or share any one or combination of the features described above with respect to the retrograde sheath 102 in FIGS. 1-6, the retrograde sheath 402 in FIGS. 7-11 and/or the retrograde sheath 702 in FIGS. 12-16. The retrograde sheath 702 may for example have a interior lumen 710 with an inner diameter of approximately 0.51 mm. In one example, the interior lumen 710 may have an inner diameter between 0.10 mm and 0.82 mm. In yet another example, the lumen diameter may be between 0.42 mm and 62 mm. In one example, the lumen inner diameter of the interior lumen 710 may be sufficiently sized to fit a common 0.035-inch (0.889 mm) diameter guidewire.

Further, as best shown in FIGS. 18A-20B, the retrograde sheath 702 may further include the connection section 708. The connection section 708 may for example have a first opening 791a and a second opening 791b and a slit 790 extending from the first opening 791a to the second opening 791b. The length of the connection section 708 (shown as CS in FIG. 18E) may for example be approximately 55.7 mm. In one example, the length CS may for example be between 25 mm and 180 mm. In another example, the length CS may be between 35 mm and 130 mm. In another example, the length CS may be between 45 mm and 85 mm. Further, a length TS from the distal end of the connection section 708 may for example be approximately 43.75 mm. In one example, the length TS may be between 20 mm and 85 mm. In another example, the length TS may be between 30 mm and 75 mm.

As shown in FIG. 19B, the connection section 708 may for example have a substantially circular cross section forming a partially enclosed inner lumen. The partially enclosed inner lumen of the connection section 708 may for example have an inner diameter of approximately 1.26 mm. In one example, the inner lumen of the connection section may for example have an inner diameter between 0.90 mm and 1.80 mm. In another example, the inner lumen may have an inner diameter between 1.00 mm and 1.50 mm.

FIG. 17C shows one example of an antegrade sheath 802 as described above. The antegrade sheath 802 may for example be analogous with or share any one or combination of the features described above with respect to the antegrade sheath 202 in FIGS. 1-6, the antegrade sheath 502 in FIGS. 7-11 and/or the antegrade sheath 802 in FIG. 12-16. In one example, the length of the antegrade sheath 802 may for example be between 50 millimeters (mm) and 200 mm. In another example, the length of the antegrade sheath 802 may for example be between 75 mm and 150 mm. In yet another example, the length of the antegrade sheath 802 may be between 85 mm and 105 mm. In another example, the length of the antegrade sheath may be approximately 95.25 mm. Further, the inner diameter of the antegrade sheath 802 lumen may be approximately 0.52 mm. In one example, the lumen may be between 0.10 mm and 0.82 mm. In yet another example, the lumen diameter may be between 0.42 mm and 62 mm. In one example, the lumen inner diameter of the antegrade sheath 802 may be sufficiently sized to fit a common 0.035-inch (0.889 mm) diameter guidewire.

Further, the outer diameter of the antegrade sheath 802 may be approximately 1.02 mm. In one example the antegrade sheath may for example have a outer diameter between 0.60 mm and 1.50 mm. In another example, the antegrade sheath 802 may for example have an outer diameter between 0.95 mm and 1.10 mm.

Additional aspects of the disclosure are described in the clauses that follow:

Clause 1. A method of performing an interventional procedure, the method comprising: retrograde placement of a needle into a femoral artery of a patient to create an incision; retrograde advancement of a first guidewire in to the femoral artery; advancing a sheath system over the guidewire and inserting a distal end of a sheath system through the incision and into the femoral artery of the patient, wherein the sheath system comprises a retrograde sheath having an antegrade sheath removably connected thereto, wherein the retrograde sheath comprises a lumen extending between a retrograde sheath distal end and a retrograde sheath proximal end and wherein the antegrade sheath comprises a lumen extending between an antegrade sheath distal end and an antegrade sheath proximal end, the sheath system further comprising connection section connecting the retrograde sheath to the antegrade sheath; antegrade advancement of a second guidewire into the femoral artery via the lumen of the antegrade sheath; separating the retrograde sheath from the antegrade sheath via the connection section; and performing an antegrade interventional procedure.

Clause 2. The method of clause 1, wherein the connection section comprises a tear away section and the retrograde sheath is separated from the antegrade sheath via the tear away section

Clause 3. The method of any one of or combination of the preceding clauses, wherein the tear away section is configured to separate one or more walls of the retrograde sheath.

Clause 4. The method of any one of or combination of the preceding clauses, wherein the retrograde sheath is removed after separation from the antegrade sheath.

Clause 5. The method of any one of or combination of the preceding clauses, wherein the retrograde sheath is removed before performing the antegrade interventional procedure.

Clause 6. The method of any one of or combination of the preceding clauses, wherein the antegrade sheath has a greater stiffness than the antegrade sheath.

Clause 7. The method of any one of or combination of the preceding clauses, further comprising separating one or more walls of the antegrade sheath via the connection section and removing the retrograde sheath while allowing the antegrade sheath to remain in the femoral artery of the patient.

Clause 8. The method of any one of or combination of the preceding clauses, wherein the antegrade sheath is separated from a concave channel in the retrograde sheath.

Clause 9. The method of any one of or combination of the preceding clauses, wherein the connection section comprises the concave channel and a separable slit for containing the antegrade sheath, wherein separating the antegrade sheath from the retrograde sheath comprises removal of the antegrade sheath from the concave channel via the separable slit.

Clause 10. The method of any one of or combination of the preceding clauses, wherein the connection section and separable slit extends along a connection section length of the retrograde sheath, wherein the antegrade sheath is separatable from the retrograde sheath along the connection section length via the separable slit.

Clause 11. A system for performing an interventional procedure, the system comprising: a retrograde sheath having an antegrade sheath removably connected thereto, wherein the retrograde sheath comprises a lumen extending between a retrograde sheath distal end and a retrograde sheath proximal end and wherein the antegrade sheath comprises a lumen extending between an antegrade sheath distal end and an antegrade sheath proximal end; a connection section connecting the retrograde sheath to the antegrade sheath wherein the connection section is configured to allow for separation of the antegrade sheath from the retrograde sheath.

Clause 12. The system of clause 11, wherein the connection section comprises a tear away section and the retrograde sheath is separated from the antegrade sheath via the tear away section.

Clause 13. The system of any one of or combination of the preceding clauses, wherein the connection section comprises a separable slit, wherein the separable slit is configured to allow the antegrade sheath to pass therethrough allowing for separation of the retrograde sheath from the antegrade sheath.

Clause 14. The system of any one of or combination of the preceding clauses, wherein the antegrade sheath is separated from a concave channel in the retrograde sheath.

Clause 15. The system of any one of or combination of the preceding clauses, wherein the connection section comprises the concave channel and a separable slit for containing the antegrade sheath, wherein separating the antegrade sheath from the retrograde sheath comprises removal of the antegrade sheath from the concave channel via the separable slit.

Clause 16. The system of any one of or combination of the preceding clauses, wherein the connection section and separable slit extends along a connection section length of the retrograde sheath, wherein the antegrade sheath is separatable from the retrograde sheath along the connection section length via the separable slit.

Clause 17. The system of any one of or combination of the preceding clauses, wherein the antegrade sheath has a greater stiffness than the antegrade sheath.

Clause 18. The system of any one of or combination of the preceding clauses, wherein a tip portion of the retrograde sheath has a greater stiffness than a body portion of the retrograde sheath.

Clause 19. The system of any one of or combination of the preceding clauses, wherein the connection section is configured to separate one or more walls of the retrograde sheath when the retrograde sheath is retracted from the antegrade sheath by a user.

Clause 20. The system of any one of or combination of the preceding clauses, wherein a portion of the antegrade sheath is configured to tear away from one or more walls of the retrograde sheath when the retrograde sheath is retracted from the antegrade sheath by a user.

While a number of example aspects and aspects have been discussed above, those of skill in the art will recognize that still further modifications, permutations, additions and sub-combinations thereof of the features of the disclosed aspects are still possible. It is therefore intended that the following appended claims and claims hereafter introduced are interpreted to include all such modifications, permutations, additions and sub-combinations as are within their true spirit and scope.

What is claimed is:

1. A method of performing an interventional procedure, the method comprising:
retrograde placement of a needle into a femoral artery of a patient to create an incision;
retrograde advancement of a first guidewire in to the femoral artery;
advancing a sheath system over the guidewire and inserting a distal end of a sheath system through the incision and into the femoral artery of the patient, wherein the sheath system comprises a retrograde sheath having an antegrade sheath removably connected thereto, wherein the retrograde sheath comprises a lumen extending between a retrograde sheath distal end and a retrograde sheath proximal end and wherein the antegrade sheath comprises a lumen extending between an antegrade sheath distal end and an antegrade sheath proximal end, the sheath system further comprising connection section connecting the retrograde sheath to the antegrade sheath;
antegrade advancement of a second guidewire into the femoral artery via the lumen of the antegrade sheath;
separating the retrograde sheath from the antegrade sheath via the connection section; and
performing an antegrade interventional procedure.

2. The method of claim 1, wherein the connection section comprises a tear away section and the retrograde sheath is separated from the antegrade sheath via the tear away section

3. The method of claim 2, wherein the tear away section is configured to separate one or more walls of the retrograde sheath.

4. The method of claim 1, wherein the retrograde sheath is removed after separation from the antegrade sheath.

5. The method of claim 4, wherein the retrograde sheath is removed before performing the antegrade interventional procedure.

6. The method of claim 1, wherein the antegrade sheath has a greater stiffness than the antegrade sheath.

7. The method of claim 1, further comprising separating one or more walls of the antegrade sheath via the connection section and removing the retrograde sheath while allowing the antegrade sheath to remain in the femoral artery of the patient.

8. The method of claim 1, wherein the antegrade sheath is separated from a concave channel in the retrograde sheath.

9. The method of claim 8, wherein the connection section comprises the concave channel and a separable slit for containing the antegrade sheath, wherein separating the antegrade sheath from the retrograde sheath comprises removal of the antegrade sheath from the concave channel via the separable slit.

10. The method of claim 9, wherein the connection section and separable slit extends along a connection section length of the retrograde sheath, wherein the antegrade sheath is separatable from the retrograde sheath along the connection section length via the separable slit.

11. A system for performing an interventional procedure, the system comprising:
a retrograde sheath having an antegrade sheath removably connected thereto, wherein the retrograde sheath comprises a lumen extending between a retrograde sheath distal end and a retrograde sheath proximal end and wherein the antegrade sheath comprises a lumen extending between an antegrade sheath distal end and an antegrade sheath proximal end;
a connection section connecting the retrograde sheath to the antegrade sheath wherein the connection section is configured to allow for separation of the antegrade sheath from the retrograde sheath.

12. The system of claim 11, wherein the connection section comprises a tear away section and the retrograde sheath is separated from the antegrade sheath via the tear away section.

13. The system of claim 11, wherein the connection section comprises a separable slit, wherein the separable slit is configured to allow the antegrade sheath to pass therethrough allowing for separation of the retrograde sheath from the antegrade sheath.

14. The system of claim 11, wherein the antegrade sheath is separated from a concave channel in the retrograde sheath.

15. The system of claim 14, wherein the connection section comprises the concave channel and a separable slit for containing the antegrade sheath, wherein separating the antegrade sheath from the retrograde sheath comprises removal of the antegrade sheath from the concave channel via the separable slit.

16. The system of claim 15, wherein the connection section and separable slit extends along a connection section length of the retrograde sheath, wherein the antegrade sheath is separatable from the retrograde sheath along the connection section length via the separable slit.

17. The system of claim 11, wherein the antegrade sheath has a greater stiffness than the antegrade sheath.

18. The system of claim 17, wherein a tip portion of the retrograde sheath has a greater stiffness than a body portion of the retrograde sheath.

19. The system of claim 11, wherein the connection section is configured to separate one or more walls of the retrograde sheath when the retrograde sheath is retracted from the antegrade sheath by a user.

20. The system of claim 11, wherein a portion of the antegrade sheath is configured to tear away from one or more walls of the retrograde sheath when the retrograde sheath is retracted from the antegrade sheath by a user.
